SEQUENCE LISTING (1 sequence)

SEQ ID NO: 1 - DNA, 11 bp, synthetic construct
  source 1..11: mol_type = other DNA
accgcagccaa

(19) United States
(12) Patent Application Publication
(10) Pub. No.: US 2024/0075082 A1
(43) Pub. Date: Mar. 7, 2024

(54) LACTIC ACID BACTERIUM AND METHOD FOR PREVENTING OR TREATING ALLERGY BY ADMINISTERING SAME

(71) Applicant: Bened Biomedical Co., Ltd., Taipei City (TW)

(72) Inventors: Ying-Chieh Tsai, Taipei City (TW); Yu-Hsuan Wei, Taipei City (TW); Chih-Chieh Hsu, Taipei City (TW); Chien-Chen Wu, Taipei City (TW)

(21) Appl. No.: 18/455,215

(22) Filed: Aug. 24, 2023

Related U.S. Application Data
(60) Provisional application No. 63/400,777, filed on Aug. 25, 2022.

(51) Int. Cl. A61K 35/747; A61K 9/00; A61P 11/00; A61P 37/08; C12N 1/20; C12N 1/36

(52) U.S. Cl. CPC A61K 35/747; A61K 9/0053; A61P 11/00; A61P 37/08; C12N 1/205; C12N 1/36; C12R 2001/225

(57) ABSTRACT

Provided is a composition including a lactic acid bacterium and a carrier thereof for prophylaxis or treatment of an allergy. The lactic acid bacterium is Lactobacillus paragasseri, such as Lactobacillus paragasseri BBM171 deposited under DSMZ Accession No. DSM 34311. Also provided is a method for preventing or treating an allergy in a subject that includes administering an effective amount of the composition of Lactobacillus paragasseri to the subject.

### BACKGROUND

### Technical Field

The present disclosure relates to a lactic acid bacterium, and more particularly relates to a lactic acid bacterium strain for prophylaxis or treatment of an allergy in a subject.

### Description of Related Art

In the past few decades, the incidences of allergic disorders, such as bronchial asthma, allergic rhinitis, and atopic dermatitis have increased dramatically, and these allergic diseases are associated with complicated mechanisms which are difficult to treat effectively even though several medications have been developed. Many medications for treating allergies are only symptomatic, and in view of the increase in the number of affected patients and the side effects that accompany long-term uses, more effective treatments have been desired.

Lactic acid bacteria (LAB) are a group of gram-positive bacteria found naturally in, for example, fermented foods such as fermented milk and fermented vegetables. LAB are named after their ability to produce lactic acid, which is an anti-bacterial substance. As such, they have been adopted in the manufacturing of fermented foods, including various forms of fermented dairy products, breads, and vegetables, and are consumed through these fermented foods for long in human history. LAB therefore have attracted a great deal of attention in that LAB have been found to exhibit valuable properties to humans and animals upon ingestion. For example, specific strains of the genus Lactobacillus have not only been recognized as alternatives for prevention or treatment of gut conditions because of their capability in regulating host's gut microbiota in recent years, but also been found to alter host's mental and physical responses for psychological stress.

In addition, it is known that some probiotics have health-promoting properties such as immunomodulation or immune tolerance to prevent or treat allergies. However, given the vast variety of probiotics and their distinct properties, effectiveness of LAB in treating allergies varies due to the properties and capabilities of different strains. Therefore, there remains space for improvement when preparing agents with advantageous immunomodulatory and anti-inflammatory activity for preventing and/or treating allergies of interest, or dietary compositions for preventing and/or treating allergies.

### SUMMARY

The present disclosure provides a composition including a lactic acid bacterium and a carrier thereof. In an embodiment, the lactic acid bacterium is Lactobacillus paragasseri. In a further embodiment, the lactic acid bacterium is Lactobacillus paragasseri BBM171, deposited under DSMZ Accession No. DSM 34311. In at least one embodiment, the lactic acid bacterium is in a form selected from the group consisting of a culture, a concentrate, a paste, a liquid, a dried product, a diluted product, and a crushed product. In an embodiment, the dried product is a spray-dried powder, a freeze-dried powder, a vacuum-dried powder, or a drum-dried powder. In at least one embodiment, the lactic acid bacterium is in a form of live or heat-inactivated bacterium. In an embodiment, the composition of the present disclosure is a dietary composition or a pharmaceutical composition.

In at least one embodiment of the present disclosure, a method for preventing or treating an allergy in a subject in need thereof is also provided. In an embodiment, the method of the present disclosure comprises administering an effective amount of Lactobacillus paragasseri to the subject in need thereof. In a further embodiment, the method of the present disclosure comprises administering an effective amount of Lactobacillus paragasseri BBM171 to the subject in need thereof.

In at least one embodiment, the lactic acid bacterium is orally administered to the subject. In an embodiment, the effective amount is at least 1×10⁶ CFU, at least 1×10⁷ CFU, at least 1×10⁸ CFU, at least 1×10⁹ CFU, at least 1×10¹⁰ CFU or at least 1×10¹¹ CFU, including 5×10⁶ CFU, 5×10⁷ CFU, 5×10⁸ CFU, 2×10⁹ CFU, 3×10⁹ CFU, 4×10⁹ CFU, 5×10⁹ CFU, 6×10⁹ CFU, 7×10⁹ CFU, 8×10⁹ CFU, 9×10⁹ CFU, 2×10¹⁰ CFU, 3×10¹⁰ CFU, 4×10¹⁰ CFU, 5×10¹⁰ CFU, 6×10¹⁰ CFU, 7×10¹⁰ CFU, 8×10¹⁰ CFU, 9×10¹⁰ CFU, 2×10¹¹ CFU, 3×10¹¹ CFU, 4×10¹¹ CFU, 5×10¹¹ CFU, 6×10¹¹ CFU, 7×10¹¹ CFU, 8×10¹¹ CFU, and 9×10¹¹ CFU, but not limited thereto. In an embodiment, the allergy is a food allergy, an insect allergy, a mold allergy, a pollen allergy, a skin allergy, or a respiratory allergy.

In at least one embodiment of the present disclosure, a serum immunoglobulin level is modulated in the subject after the administration. In a further embodiment, the serum immunoglobulin level is a level of at least one of IgE, IgG1 and IgG2a.

In at least one embodiment, a level of airway inflammation is decreased in the subject after the administration. In a further embodiment, an inflammatory cell infiltration is reduced in the subject. In some embodiments, a mucus production in the respiratory tract is reduced in the subject.

In at least one embodiment, a level of inflammatory cell accumulation in bronchoalveolar lavage fluid is decreased in the subject after the administration. In a further embodiment, the inflammatory cell is a leukocyte. In still a further embodiment, the inflammatory cell is an eosinophil.

In at least one embodiment, a level of cytokine is altered in the subject after the administration. In a further embodiment, the cytokine is a proinflammatory cytokine or an anti-inflammatory cytokine. In yet a further embodiment, the cytokine is a Th17 cytokine, a Treg cytokine, a Th1 cytokine or a Th2 cytokine. In some embodiments, the Th17 cytokine is IL-17A. In some embodiments, the Treg cytokine is IL-10. In some embodiments, the Th1 cytokine is at least one of IFN-γ and IL-12. In some embodiments, the Th2 cytokine is at least one of IL-4, IL-5, and IL-13.

In at least one embodiment, the cytokine alteration of the present disclosure modulates at least one of a Th1 immune response and a Th2 immune response. In some embodiments, the cytokine alteration modulates the immune response towards Th1 in the subject.

### BRIEF DESCRIPTION OF DRAWINGS

The present disclosure can be more understood by reading the following descriptions of the embodiments, with reference made to one or more of the accompanying drawings below.

FIG. 1 shows an electrophoresis photograph showing the random amplified polymorphic DNA (RAPD) profiles of two Lactobacillus paragasseri strains. Lane M, DNA ladder (250 to 10,000 bp); Lane 1, Lactobacillus paragasseri JCM 5343^{T}; Lane 2, Lactobacillus paragasseri BBM171.

FIGS. 2A to 2D show effects of L. paragasseri BBM171 on the serum immunoglobulin levels in OVA-induced allergic mice. FIG. 2A shows levels of total IgE in four different mice groups. FIG. 2B shows levels of OVA-specific IgE in four different mice groups. FIG. 2C shows levels of OVA-specific IgG1 in four different mice groups. FIG. 2D shows levels of OVA-specific IgG2a in four different mice groups. Data are expressed as mean±SEM and analyzed by one-way ANOVA with Tukey's post hoc test. *** denotes significant differences at P<0.001 compared with the CON group; ^{#} denotes P<0.05 and ^{##} denotes P<0.01 compared with the OVA group.

FIGS. 3A to 3D show that oral administration of L. paragasseri BBM171 reduced inflammatory cell infiltration and amount of mucus in the lung tissues of OVA-induced allergic mice. FIG. 3A shows the result of Hematoxylin and Eosin (H&E) staining. FIG. 3B shows the results of periodic acid-Schiff (PAS) staining. FIG. 3C depicts the inflammation scores representing the semi-quantitative results of airway inflammatory cells stained with H&E. FIG. 3D shows the semi-quantitative results of mucus secretion stained with PAS. Scale bar=50 μm in FIGS. 3A and 3B. Data are expressed as mean±SEM and analyzed by one-way ANOVA with Tukey's post hoc test. ** denotes significant differences at P<0.001 compared with the CON group. ^{#} and ^{##} denote P<0.05 and P<0.01, respectively, compared with the OVA group.

FIGS. 4A and 4B show effects of L. paragasseri BBM171 intervention on the distribution of immune cells in BALF. In four mice groups, FIG. 4A shows total count of leukocytes, and FIG. 4B shows cell counts of eosinophils. Data are expressed as mean±SEM and analyzed by one-way ANOVA with Tukey's post hoc test. * denotes significant differences at P<0.001 compared with the CON group. ^{#} and ^{##} denote P<0.05 and P<0.01, respectively, compared with the OVA group.

FIGS. 5A to 5F show effects of L. paragasseri BBM171 on the production of Th1- and Th2-related cytokines IFN-γ, IL-12, IL-4, IL-5, IL-13 and IL-4/IL-12 ratio, respectively, in the BALF determined by ELISA. Data are expressed as mean±SEM and analyzed by one-way ANOVA with Tukey's post hoc test. *, and *** denote significant differences at P<0.05, P<0.01 and P<0.001 compared with the CON group, respectively. ^{#} denotes P<0.05 compared with the OVA group.

FIGS. 6A to 6H show effects of L. paragasseri BBM171 on the production of T-cell cytokines IFN-γ, IL-12, IL-2, IL-4, IL-5, IL-13, IL-17A, and IL-10, respectively, produced by splenocytes from OVA-sensitized mice in response to OVA. Data are expressed as mean f SEM and analyzed by one-way ANOVA with Tukey's post hoc test. and ***denote P<0.01 and P<0.001, respectively, compared with the CON group. ^{#}, ^{##} and ^{###} denote P<0.05, P<0.01, and P<0.001, respectively, compared with the OVA group. $ denotes P<0.05 between Live and Heat-inactivated groups.

### DETAILED DESCRIPTION

The following examples are used for illustrating the present disclosure. A person skilled in the art can easily conceive the other effects of the present disclosure, based on the disclosure of the specification. It will be apparent that one or more embodiments may be practiced without specific details. The present disclosure can also be implemented or applied as described in different examples. It is possible to modify or alter the following examples for carrying out this disclosure without contravening its scope for different applications. Titles or subtitles may be used in this disclosure for the convenience of a reader, which shall have no influence on the scope of the present disclosure.

In this disclosure, all terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

As used in this disclosure, the singular forms “a,” “an,” and “the” include plural referents unless expressly and unequivocally limited to one referent. The term “or” is used interchangeably with the term “and/or” unless the context clearly indicates otherwise.

Also, when a part “includes” or “comprises” a component or a step, unless there is a particular description contrary thereto, the part can further include other components or other steps, not excluding the others.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the term “carrier” may comprise, but is not limited to, a solvent, a dispersion medium, a coating, an antibacterial and antifungal agent, or an isotonic and absorption delaying agent, etc. which is suitable for pharmaceutical administration. The pharmaceutical composition comprising a carrier can be formulated into dosage forms for different administration routes utilizing conventional methods. The carrier in a pharmaceutical composition is compatible with the active ingredient of the composition (and capable of stabilizing the active ingredient) and not deleterious to the subject to be treated.

The phrase “an effective amount” refers to the amount of an active ingredient that is required to result in a reduction, inhibition, or prevention of an allergy in a subject. An effective amount will vary, as recognized by those skilled in the art, depending on routes of administration, excipient usage, and the possibility of co-usage with other therapeutic treatment.

In the context of the present disclosure, the term “immune response” or “immunological response” means, but is not limited to, the development of a cellular and/or antibody-mediated immune response. Usually, an immune or immunological response includes, but is not limited to, one or more of the following effects: the production or activation of antibodies, B cells, helper T cells, suppressor T cells, and/or cytotoxic T cells, directed specifically to an antigen or antigens.

As used herein, the terms “subject” and “individual” may be interchangeable and refer to an animal, e.g., a mammal including the human species. The term “subject” is intended to refer to both the male and female gender unless one gender is specifically indicated. Non-limiting examples of non-human animal subjects include: rodents such as mice, rats, hamsters, and guinea pigs; rabbits; dogs; cats; sheep; pigs; goats; cattle; horses; and non-human primates such as apes and monkeys.

As used herein, the term “prophylactic,” “preventing” or “prevention” refers to preventive or avoidance measures for a disease or symptoms or conditions of a disease, which include but are not limited to applying or administering one or more active agents to a subject who has not yet been diagnosed as a patient suffering from the disease or the symptoms or conditions of the disease but may be susceptible or prone to the disease, e.g., an allergy. The purpose of the preventive measures is to avoid, prevent, or postpone the occurrence of the disease or the symptoms or conditions of the disease.

As used herein, the term “treat,” “treating” or “treatment” refers to the application or administration of one or more active agents to a subject afflicted with a disorder, a symptom or a condition of a disease, or a progression of the disease, with the purpose to cure, heal, relieve, alleviate, alter, remedy, ameliorate, improve, or affect the disorder, the symptom or the condition of the disease, the disabilities induced by the disease, or the progression of the disease.

The present disclosure provides a lactic acid bacterium, a composition, and a method for preventing or treating an allergy. As used herein, the term “allergy” represents all different types of allergies, which can be categorized by the allergens that induce an allergy such as food allergy, insect allergy, mold allergy or pollen allergy, or by the part of body that manifests the allergic symptom, such as skin allergy or respiratory allergy. Respiratory allergy, or allergic airway disorders, such as allergic rhinitis (AR) and asthma, are chronic airway inflammatory diseases. The allergy types, symptoms, and diseases to which the agents of the present disclosure for preventing and/or treating allergies can be applied are not particularly limited; however, examples include type I to type IV allergies, food allergies, pollen allergies, atopic dermatitis, bronchial asthma, allergic conjunctivitis, allergic rhinitis, allergic gastroenteritis, anaphylactic reactions, drug allergies, urticaria, serum sickness, hemolytic anemia, contact dermatitis, myasthenia gravis, Goodpasture's syndrome, and glomerulonephritis.

In this disclosure, applicable allergens are not particularly limited either; examples include food (wheat, barley, oats, rye, buckwheat, eggs, milk, cheese, peanuts, rice, corn, foxtail millet, proso millet, Japanese millet, soy beans, potatoes, yams, garlic, onions, carrots, parsleys, celeries, tomatoes, oranges, peaches, apples, kiwi fruit, melons, strawberries, bananas, walnuts, sesame, matsutake mushrooms, abalones, squids, salmon caviars, shrimps, crabs, salmons, mackerels, horse mackerels, sardines, cods, squids, octopuses, scallops, beef, chicken, pork, gelatin, etc.), animals (dogs, cats, mice, rats, pigeons, and such, and their skin, hair, feces, feather, etc.), insects (moths, butterflies, chironomids, hornets, and such, and their secretion products and scales), ticks, parasites (Anisakis, ascarids, etc.), plants (cedars, cypresses, ragweeds, gramineous plants, mugwort, lacquer trees, alders, and such, and the pollens, saps, and such of these plants), molds, dust, house dust, rubber, metals, chemical substances, and pharmaceuticals.

The immune response of type 2 T helper cells (Th2) is a mechanism involved in allergic inflammation, which involves the secretion of a range of cytokines, including interleukin (IL)-4, IL-5, and IL-13. On the other hand, type 1 T helper cell (Th1)-driven cytokines such as interferon-gamma (IFN-γ), IL-2, and IL-12 can block the production of Th2 cells. Differential expression of the balanced relationship between Th1 and Th2 in the immune system involves in the development of allergic diseases. In addition, Th17 and regulatory T (Treg) cells have been reported to play a role in the pathogenesis of allergic airway inflammation. Th17 cells are characterized by the secretion of IL-17, which mediates eosinophilic airway inflammation. The suppression of Th2 cell-driven inflammatory responses by Treg cells plays a role in the development of asthma tolerance. As such, an imbalance in Th1/Th2 and Th17/Treg cell ratios leads to an inflammatory response in antigen-induced airway inflammation. The present disclosure provides a method to modulate Th1 and Th2 immune responses, so as to adjust cytokine and immunoglobulin secretion in a subject, thereby preventing and/or treating an allergy in a subject in need thereof.

### Examples

Exemplary embodiments of the present disclosure are further described in the following examples, which should not be construed to limit the scope of the present disclosure.

### Materials and Methods

Preparation of Lactobacillus paragasseri (L. paragasseri) BBM171

L. paragasseri BBM171 (DSM 34311, also referred to herein as BBM171) was isolated from breast milk, and routinely cultivated in De Man, Rogosa and Sharpe (MRS) broth (Becton, Dickinson and Company, Sparks, MD, USA) at 37° C. for 18 to 20 hours, and then harvested by centrifugation at 6000×g for 10 min.

L. paragasseri BBM171 has been deposited under Budapest Treaty at Leibniz Institute DSMZ-German Collection of Microorganisms and Cell Cultures (Inhoffenstr. 7 B, D-38124 Braunschweig, Germany) on Jun. 21, 2022 and has been given the DSMZ Accession No. DSM 34311 by the International Depositary Authority. This biological material was subjected to the viability test and passed.

For live L. paragasseri BBM171 preparation, pelleted bacteria were washed twice with sterile phosphate-buffered saline (PBS) and resuspended to attain a final concentration of 5×10⁸ colony-forming units (CFUs)/mL in PBS before oral administration.

For the preparation of heat-inactivated L. paragasseri BBM171, the pellet was resuspended to a final concentration of approximately 5×10⁸ CFU/mL in PBS and heat-inactivated at 100° C. for 20 min. The heat-inactivated bacterial solution was aliquoted into tubes and stored at −20° C. until use.

### Animals

Sexually mature female BALB/c mice (6 to 8 weeks old) were supplied by BioLASCO Taiwan Co., Ltd. The animals were maintained in an individually ventilated cage (IVC) system under a 12-hour light:12-hour dark cycle. Food and water were provided ad libitum. The use of animals and procedures for animal handling and treatments were approved by the Institutional Animal Use and Care Committee (IACUC 1060606) at the National Yang Ming Chiao Tung University.

### Mouse Model of Allergic Airway Inflammation

An ovalbumin (OVA)-induced airway allergic mouse model was used according to previously published study (Liu Y. W. et al., PLoS ONE 9 (6): e100105), with some modifications, to assess the effect of BBM171. For instance, mice were randomly divided into four groups (n=8 per group): healthy control mice (CON), OVA-induced allergic mice (OVA), allergic mice supplemented with live BBM171 (Live), and allergic mice supplemented with heat-inactivated BBM171 (Heat-inactivated). Mice in the Live and Heat-inactivated groups were orally administered with live BBM171 and heat-inactivated BBM171 in 200 μL phosphate-buffered saline (PBS) (10⁸ CFU/mouse/day), respectively, from day 1 to the last challenge day (day 48), while the CON and OVA groups were orally administered with 200 μL PBS per day. Mice in the OVA, Live, and Heat-inactivated groups were sensitized by intraperitoneal injection with 200 μL aluminum hydroxide (Al(OH)₃) (Pierce Biotechnology, Rockford, IL, USA) containing 20 μg OVA on days 7, 21, and 35. On days 46, 47, and 48, mice in the OVA, Live, and Heat-inactivated groups were challenged intranasally with 100 μg OVA solution (in 40 μL PBS) under anesthesia with pentobarbital. The CON group received Al(OH)₃ only, when sensitized, and PBS during the challenge period. On day 49, all mice were sacrificed, and bronchoalveolar lavage fluid (BALF) and blood samples were collected for further analyses.

### Immunoglobulin Measurements

Immunoglobulins (Igs) in the blood were analyzed using enzyme-linked immunosorbent assay (ELISA) kits according to the manufacturer's instructions. The levels of total IgE and OVA-specific IgE, OVA-specific IgG1, and OVA-specific IgG2a were measured using commercial ELISA kits (Bethyl Laboratory Inc., Montgomery, TX, USA, for total IgE and Alpha Diagnostic International Inc., San Antonio, TX, USA, for OVA-specific Igs).

### Histological Analysis

Histopathological staining of lung tissues was performed and analyzed according to standard procedures (Jiang X. H. et al., Allergy, asthma, and clinical immunology: Official Journal of the Canadian Society of Allergy and Clinical Immunology 16:59). After sacrifice, mice lung tissues were fixed in 10% formaldehyde solution. The specimens were then dehydrated using ethyl alcohol and xylene. Tissues were embedded in paraffin and sliced into 5-μm thick sections. The sections were stained with hematoxylin and eosin (H&E) to observe inflammatory cell infiltration, and the inflammation score was evaluated based on the amount of inflammatory cell infiltration around the airway according on the following: no infiltration (0 score); a little (1 score); more (2 scores); a large number, less than a group (3 scores); and a large number of groups (4 scores). In addition, the distribution of goblet cells and mucus secretion were determined using periodic acid-Schiff (PAS) staining and scored based on the presence of airway goblet cells according to the following: no goblet cell (0 score); less than 25% (1 score); 25% to 50% (2 scores); 50% to 75% (3 scores); and more than 75% (4 scores).

### Bronchoalveolar Lavage Fluid (BALF) Collection and Cell Count

The trachea was exposed, and the airways were lavaged twice with 1 mL of Hanks' balanced salt solution (HBSS) using a tracheal cannula. Cells in BALF were collected by centrifugation (400×g, 4° C., 10 min), and the fluid was stored at −80° C. for cytokine analysis. The collected cells from the BALF were resuspended in HBSS containing 2% fetal bovine serum, and the numbers of lymphocytes and eosinophils in the BALF were analyzed and calculated using a cytometer (Sysmex XT-1800iV).

Ex Vivo Stimulation of Splenocytes Isolated from OVA-Sensitized Mice

Mice (n=4) were sensitized by intraperitoneal injection of 200 μL of Al(OH)₃ containing 50 μg OVA on days 0 and 14. On day 21, the mice were sacrificed, and their splenocytes were isolated, pulled, and adjusted to a concentration of 2×10⁶ cells/mL. Splenocytes were re-stimulated with OVA (100 μg/mL) in a medium containing live or heat-inactivated L. paragasseri BBM171 at 2×10⁷ cells for 24 h. The culture medium was collected for cytokine measurements.

### Cytokine Measurements

Cytokines, including IL-2, IL-4, IL-5, IL-12, IL-13, IFN-γ, IL-17A, and IL-10, were measured using commercial ELISA kits (R&D Systems, Minneapolis, MN, USA).

### Statistical Analysis

In the present disclosure, data are expressed as mean±standard error of the mean (SEM). Differences between means were tested for statistical significance using one-way analysis of variance (ANOVA) followed by Tukey's post hoc test. Statistical significance was set at P<0.05.

### Example 1. Genetic Typing of Lactobacillus paragasseri BBM171

L. paragasseri BBM171 (also referred to as BBM171) was isolated from breast milk. To identify the species of BBM171, 16S rRNA and pheS gene sequences were analyzed by direct sequencing of PCR-amplified products. Genomic DNA extraction, PCR mediated amplification, purification of the PCR products, and sequencing of the purified PCR products were carried out using methods known in the art.

The resulting sequence was put into the alignment software provided online by the National Center for Biotechnology Information (NCBI), aligned manually and compared with representative 16S rRNA or pheS gene sequences of organisms belonging to the Lactobacillus, respectively. For comparison, 16S rRNA and pheS gene sequences were also obtained from the database provided online by the NCBI.

As a result of this analysis, Table 1 lists the organisms having the highest similarity values of 16S rRNA and pheS DNA sequences when compared to the 16S rRNA and pheS gene sequence of BBM171.

The comparison results of 16S rRNA gene and pheS gene indicate that BBM171 belongs to the species of Lactobacillus paragasseri.

TABLE 1
Comparison between 16S rRNA (A) and pheS (B) Gene Sequences

| | % 16S rRNA | % pheS |
|---|---|---|
| | gene sequence | gene sequence |
| | similarity to | similarity to |
| Strain (GenBank No.) | BBM171 | BBM171 |
| Lactobacillus paragasseri | 99.76 | 98.78 |
| JCM 5343^{T} | | |
| (AP018549.1) | | |
| Lactobacillus gasseri | | |
| ATCC 33323^{T} | 99.65 | 95.41 |
| (CP000413.1) | | |
| Lactobacillus taiwanensis | | |
| DSM 21401^{T} | 99.53 | 92.66 |
| (GCA_001436695.1_01775) | | |

### Example 2. Identification of Lactobacillus paragasseri BBM171 by RAPD-PCR

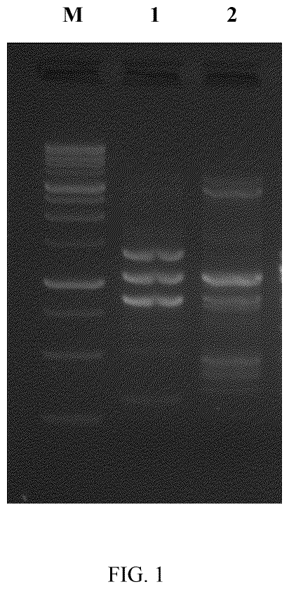

The RAPD profiles of BBM171 and Lactobacillus paragasseri JCM 5343^{T} were compared. PCR was carried out under the condition indicated in Table 2 using a random primer having a sequence of 5′-ACCGCAGCCAA-3′ (SEQ ID NO. 1). DNA respectively extracted from these strains were used as templates. The obtained amplification products were electrophoresed and the patterns were compared in FIG. 1. As shown in FIG. 1, lane M represents DNA ladder (250 to 10,000 bp), lane 1 represents Lactobacillus paragasseri JCM 5343^{T} and lane 2 represents Lactobacillus paragasseri BBM171. The results indicated that the amplified products of BBM171 had patterns different from that of Lactobacillus paragasseri type strain and that BBM171 harbored a characteristic PCR-fingerprinting deduced from its genome, representing that BBM171 is a novel strain.

TABLE 2
Composition of the PCR reaction solution (25 μL)

| Component | Volume | |
|---|---|---|
| ddH₂O | 17.9 | μL |
| 10X PCR Buffer | 2.5 | μL |
| dNTP Mix (2.5 mM) | 2.0 | μL |
| MgCl₂ (25 mM) | 1.0 | μL |
| Primer | 0.4 | μL |
| rTaq | 0.2 | μL |
| DNA template (10 μM) | 1.0 | μL |

PCR Conditions: 94° C., 2 min.; 5 cycles (94° C., 30 sec.; 36° C., 1 min.; 72° C., 1.5 min.); 30 cycles (94° C., 20 sec.; 36° C., 30 sec.; 72° C., 1.5 min.); 72° C., 3 min.

### Example 3. Analytical Profile Index (API) of Lactobacillus paragasseri (L. paragasseri) BBM171

Lactobacillus paragasseri (L. paragasseri) BBM171 of the present disclosure was characterized and analyzed using API 50 CHL kit (bioMerieux, France) for its sugar utilization profile, and the results are shown in Table 3.

TABLE 3
Fermentation Test Results of L. paragasseri BBM171

| No. | Carbohydrates substrate | BBM171 |
|---|---|---|
| 0 | CONTROL | − |
| 1 | Glycerol | − |
| 2 | Erythritol | − |
| 3 | D-Arabinose | − |
| 4 | L-Arabinose | − |
| 5 | D-Ribose | − |
| 6 | D-Xylose | − |
| 7 | L-Xylose | − |
| 8 | D-Adonitol | − |
| 9 | Methyl-β-D- | − |
| | Xylopyranoside | |
| 10 | D-Galactose | + |
| 11 | D-Glucose | + |
| 12 | D-Fructose | + |
| 13 | D-Mannose | + |
| 14 | L-Sorbose | − |
| 15 | L-Rhamnose | − |
| 16 | Dulcitol | − |
| 17 | Inositol | − |
| 18 | D-Mannitol | + |
| 19 | D-Sorbitol | + |
| 20 | Methyl-α-D- | − |
| | mannopyranoside | |
| 21 | Methyl-α-D- | − |
| | glucopyranoside | |
| 22 | N-Acetyl glucosamine | + |
| 23 | Amygdalin | + |
| 24 | Arbutin | + |
| 25 | Esculin ferric citrate | + |
| 26 | Salicin | + |
| 27 | D-Cellobiose | + |
| 28 | D-Maltose | + |
| 29 | D-Lactose | + |
| | (bovine origin) | |
| 30 | D-Melibiose | + |
| 31 | D-Saccharose (sucrose) | + |
| 32 | D-Trehalose | + |
| 33 | Inulin | − |
| 34 | D-Melezitose | + |
| 35 | D-Raffinose | − |
| 36 | Amidon (starch) | + |
| 37 | Glycogen | − |
| 38 | Xylitol | − |
| 39 | Gentiobiose | + |
| 40 | D-Turanose | + |
| 41 | D-Lyxose | − |
| 42 | D-Tagatose | + |
| 43 | D-Fucose | − |
| 44 | L-Fucose | − |
| 45 | D-Arabitol | − |
| 46 | L-Arabitol | − |
| 47 | Potassium gluconate | − |
| 48 | Potassium 2- | − |
| | ketogluconate | |
| 49 | Potassium 5- | − |
| | ketogluconate | |

### Example 4. Oral Administration of L. Paragasseri BBM171 Modulated the Production of Serum Igs in OVA-Induced Allergic Mice

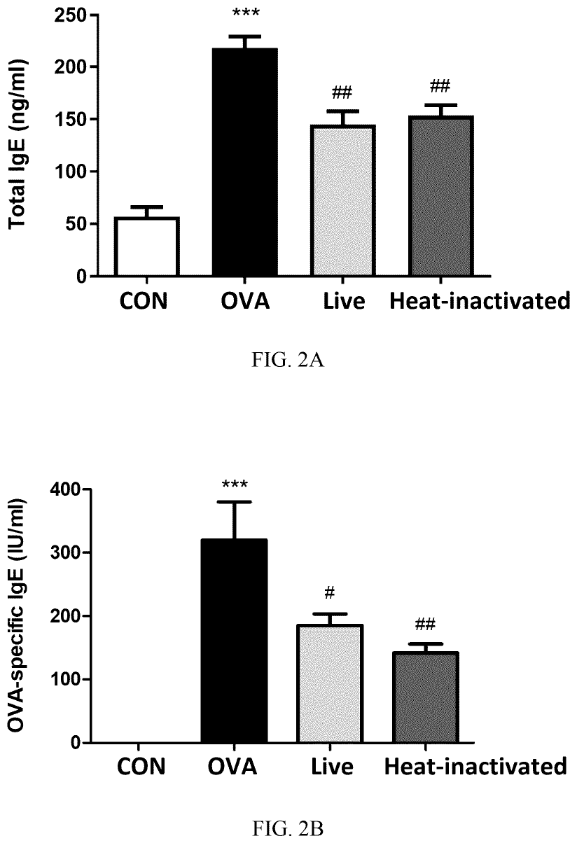

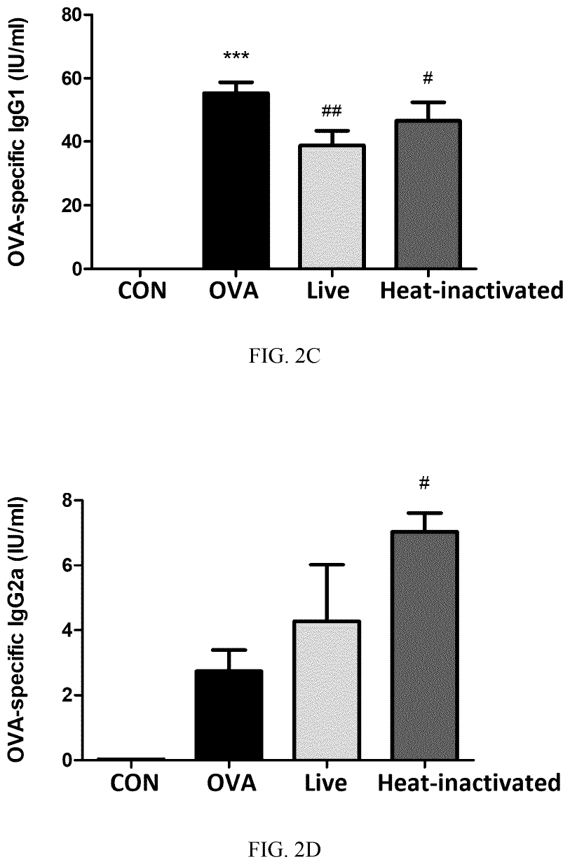

Total IgE and OVA-specific immunoglobulins (Igs) were assessed to evaluate individual responses to allergen exposure. As shown in FIG. 2A, the serum total IgE level was significantly higher in the OVA group than in the CON group (P<0.001). Live (treated with oral live L. paragasseri BBM171) and Heat-inactivated (treated with oral heat-inactivated L. paragasseri BBM171) groups had lower serum total IgE levels than the OVA group (P<0.01).

Compared with the CON group, the levels of OVA-specific IgE and IgG1 were higher in the OVA group, which were reduced in the groups receiving supplementation of live or heat-inactivated L. paragasseri BBM171, as shown in FIGS. 2B and 2C.

In addition, mice receiving supplementation of live or heat-inactivated L. paragasseri BBM171 had increased serum OVA-specific IgG2a levels, the Th1-type immunoglobulin, compared to those in the OVA group, as shown in FIG. 2D.

### Example 5. L. paragasseri BBM171 Ameliorated Airway Inflammation in OVA-Induced Allergic Mice

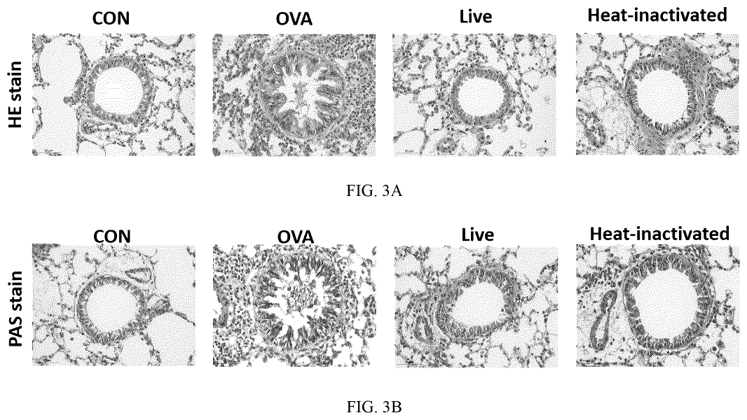

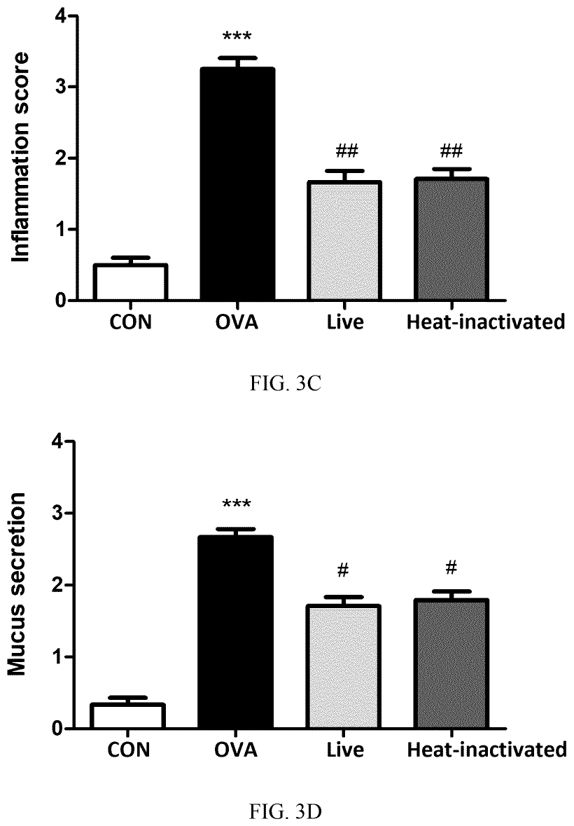

Inflammatory cell infiltration and mucus production in the lung tissues were analyzed to assess the development of airway inflammation. Compared to the CON group, histological analysis and PAS staining of lung sections revealed significant increases in inflammatory cell infiltration and mucus production in the OVA group, as shown in FIGS. 3A and 3B. However, these effects were alleviated by the supplementation of live or heat-inactivated L. paragasseri BBM171. Semi-quantitative results of airway inflammatory cell infiltration and mucus secretion shown in FIGS. 3C and 3D indicate that both airway inflammatory cell infiltration and mucus secretion significantly reduced in mice groups treated with live or heat-inactivated L. paragasseri BBM171.

### Example 6. L. paragasseri BBM171 Reduced the Accumulation of Inflammatory Cells in the BALF

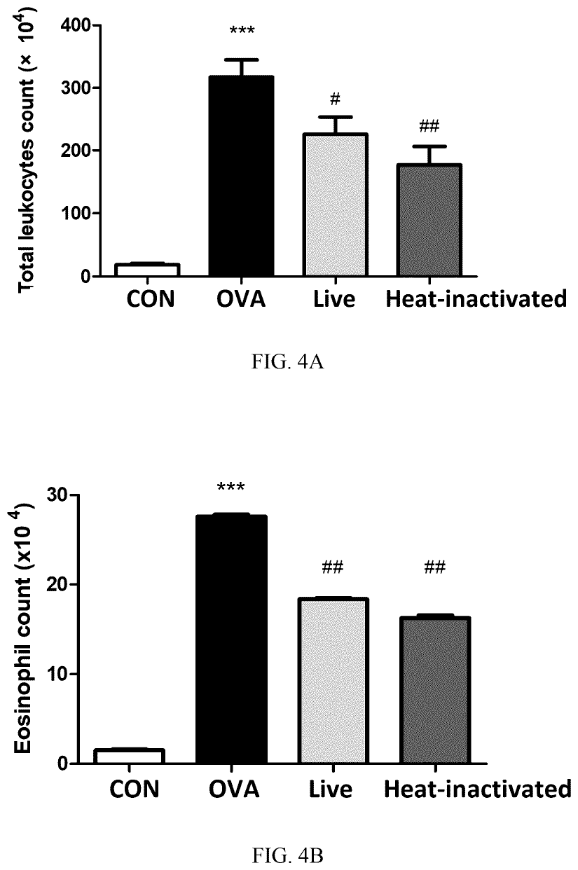

To assess the effect of L. paragasseri BBM171 on OVA-induced recruitment and accumulation of inflammatory cells in the lungs, the levels of leukocytes and eosinophils in BALF were measured. Compared to the CON group, the levels of total leukocytes and eosinophils were significantly higher in the OVA group, as shown in FIGS. 4A and 4B. However, also shown in FIGS. 4A and 4B, supplementation of live or heat-inactivated L. paragasseri BBM171 reversed the increase of inflammatory cells in OVA-induced allergic mice, where both total leukocytes count and eosinophil count were reduced significantly compared to the OVA group.

### Example 7. L. paragasseri BBM171 Modulated the Th1/Th2 Responses in the Lungs of OVA-Induced Allergic Mice

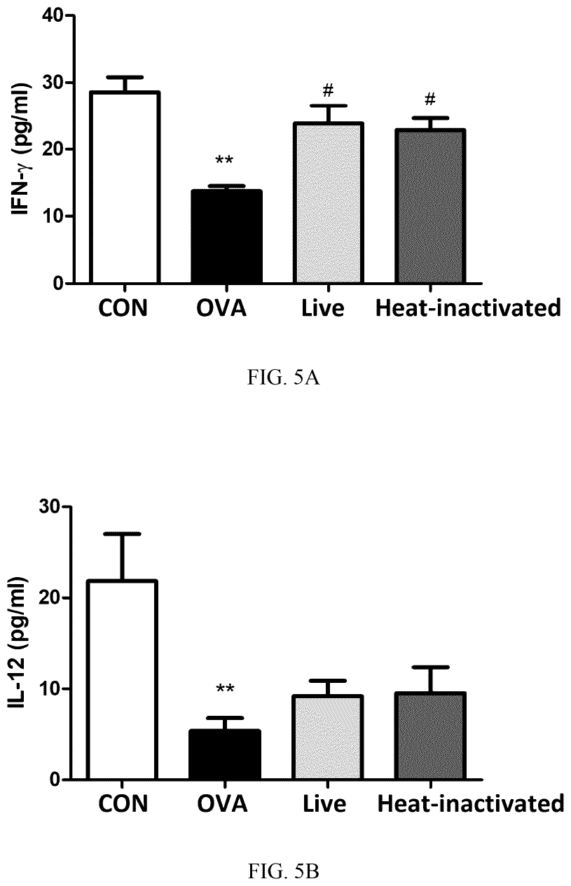

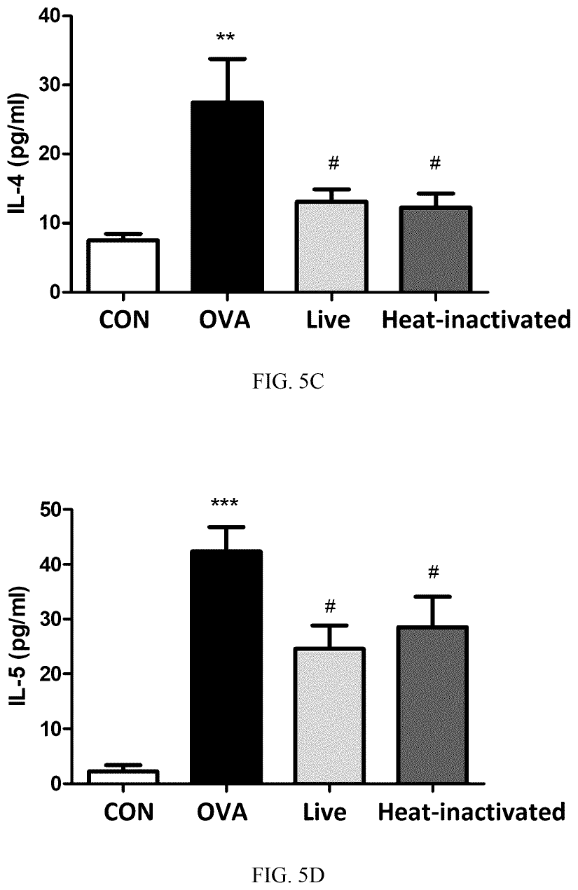

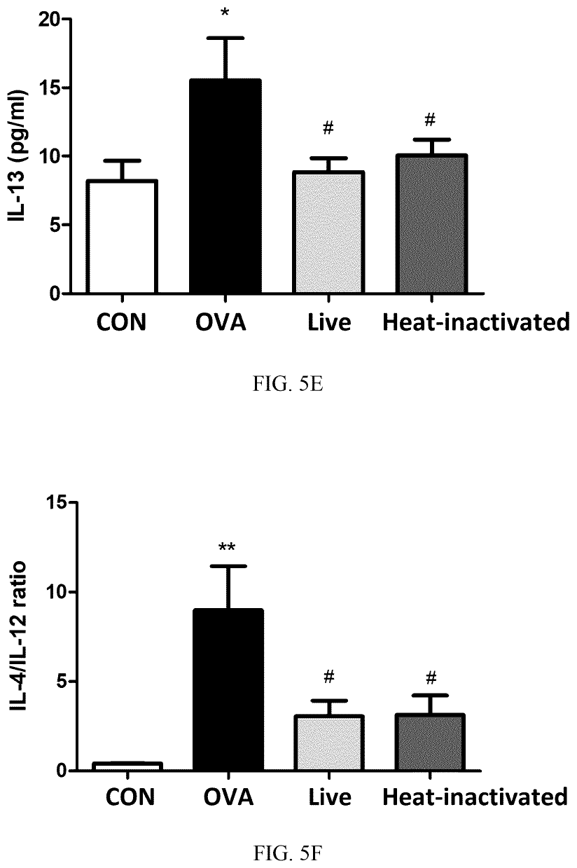

The concentrations of Th1 and Th2 cytokines in BALF were measured in four different mice groups. Compared to the CON group, the levels of classic Th1 cytokines (IFN-γ and IL-12) were significantly decreased in the OVA group, as shown in FIGS. 5A and 5B. Supplementation with live or heat-inactivated BBM171 significantly attenuated the decrease of IFN-γ.

In addition, compared to the CON group, the levels of Th2 cytokines (IL-4, IL-5, and IL-13) were significantly elevated in the OVA group, as shown in FIGS. 5C to 5E. The increases of these cytokines were reversed by supplementation of live or heat-inactivated L. paragasseri BBM171.

Furthermore, compared to the CON group, the IL-4/IL-12 ratio was significantly higher in the OVA group, suggesting Th2 dominance in the OVA group, as shown in FIG. 5F. Supplementation with live or heat-inactivated L. paragasseri BBM171 significantly decreased the IL-4/IL-12 ratio in the BALF of OVA-induced allergic mice, indicating that L. paragasseri BBM171 modulates the immune response toward Th1.

### Example 8. Effects of L. paragasseri BBM171 on the Cytokine Profile in Splenocytes from OVA-Induced Allergic Mice

To further evaluate the immunomodulatory effects of live and heat-inactivated L. paragasseri BBM171 on T cell responses, an ex vivo experiment was performed, following the method described above. In the ex vivo experiment, splenocytes from OVA-sensitized mice were isolated, collected, divided into four groups for different treatments, and incubated for 24 hours to analyze cytokine levels in the supernatant.

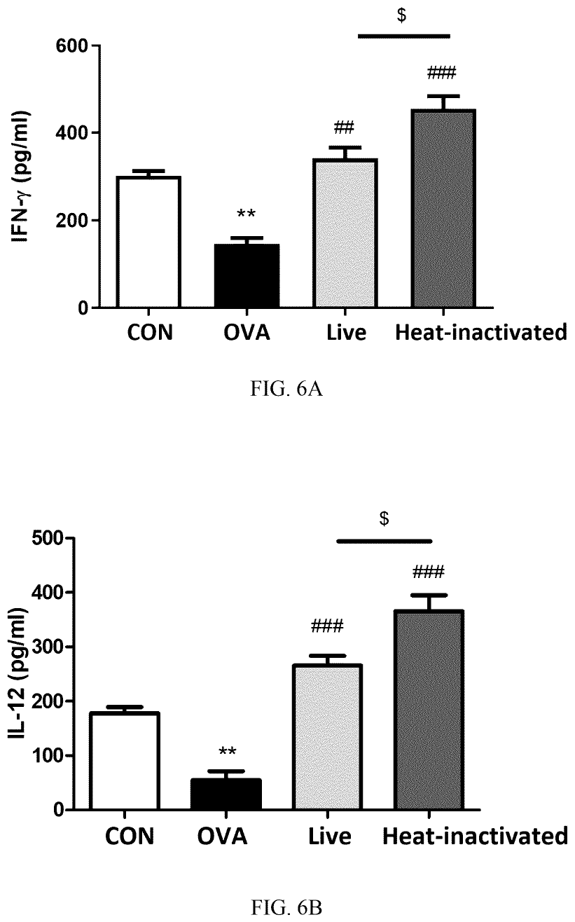

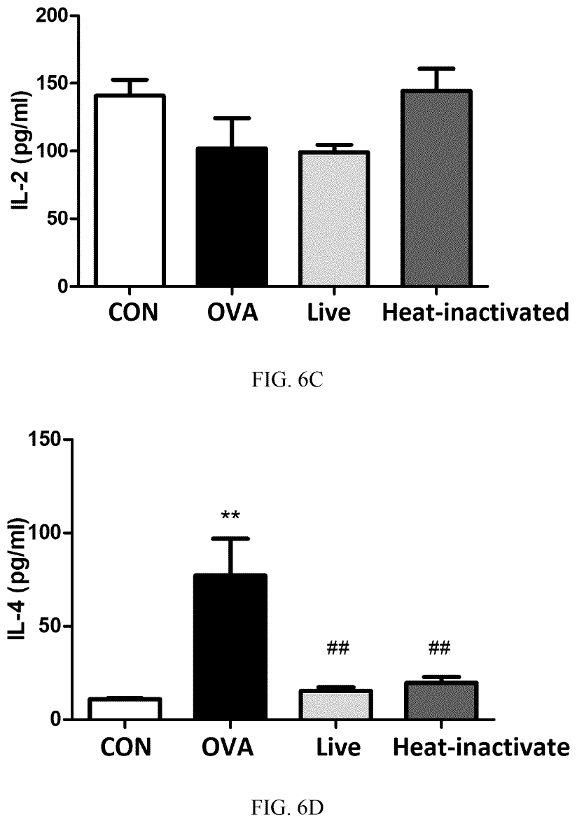

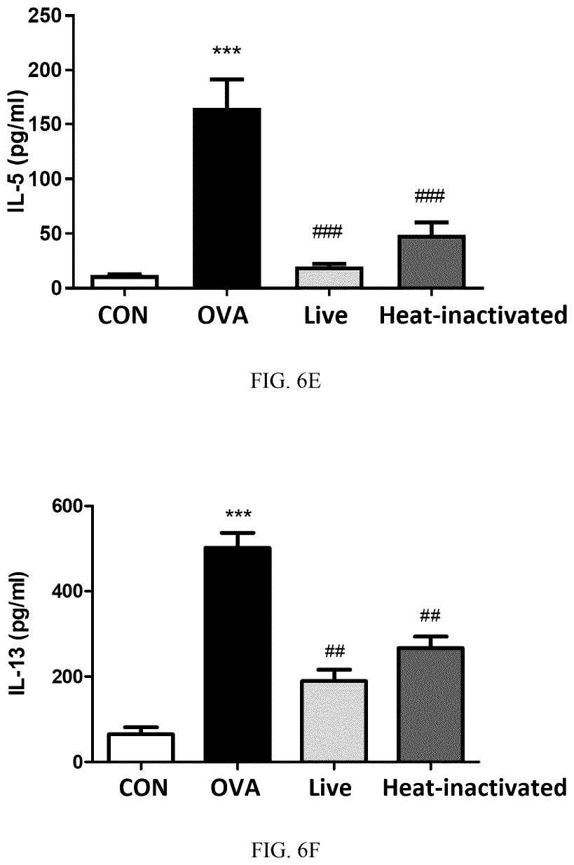

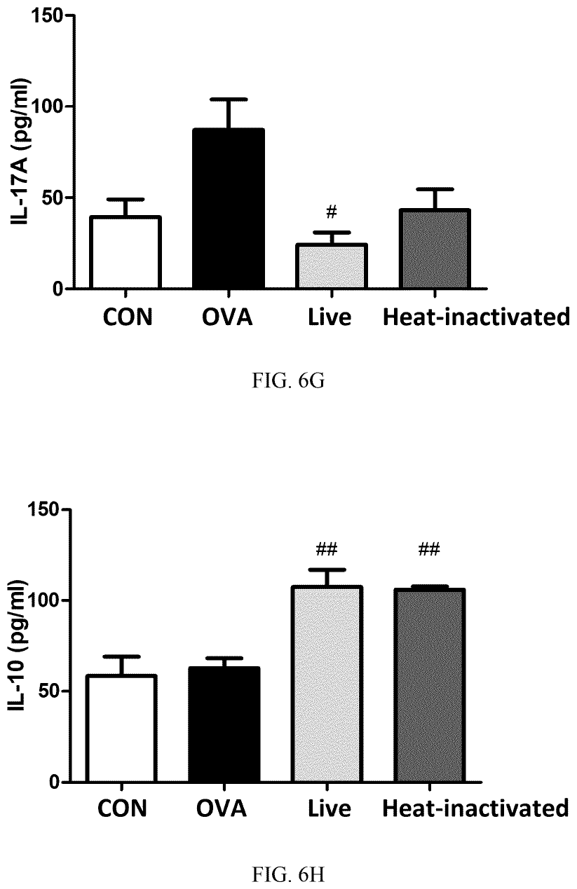

Compared to the CON group where only PBS was treated, re-stimulation with OVA reduced the production of Th1-driven IFN-γ (FIG. 6A) and IL-12 (FIG. 6B), which were significantly increased by the addition of live or heat-inactivated L. paragasseri BBM171.

For IL-2, a Th1-driven cytokine, there was no significant difference among the four different treatment groups (FIG. 6C). Compared with the CON group, re-stimulation with OVA increased the production of Th2-driven cytokines IL-4 (FIG. 6D), IL-5 (FIG. 6E), and IL-13 (FIG. 6F), which were reversed by the addition of live or heat-inactivated L. paragasseri BBM171.

In addition, the level of the cardinal Th17 cytokine, IL-17A, was significantly lower in the L. paragasseri BBM171 group than in the OVA group (FIG. 6G). Moreover, both live and heat-inactivated L. paragasseri BBM171 regulated Treg responses to increase IL-10 production in OVA-treated splenocytes (FIG. 6H).

These results indicate that L. paragasseri BBM171 effectively modulates the Th1/Th2 balance in splenocyte culture in response to OVA challenge, and that L. paragasseri BBM171 reduces the levels of proinflammatory cytokine IL-17A while increasing those of the anti-inflammatory cytokine IL-10.

While some of the embodiments of the present disclosure have been described in detail in the above, it is, however, possible for those of ordinary skill in the art to make various modifications and changes to the embodiments shown without substantially departing from the teaching of the present disclosure. Such modifications and changes are encompassed in the scope of the present disclosure as set forth in the appended claims.

What is claimed is:

1. A composition comprising a lactic acid bacterium and a carrier thereof, wherein the lactic acid bacterium is Lactobacillus paragasseri in an amount of at least 1×10⁶ colony-forming unit (CFU) for oral delivery.

2. The composition of claim 1, wherein the lactic acid bacterium is Lactobacillus paragasseri BBM171 deposited under DSMZ Accession No. DSM 34311.

3. The composition of claim 1, being in a form selected from the group consisting of a culture, a concentrate, a paste, a liquid, a dried product, a diluted product, and a crushed product.

4. The composition of claim 3, wherein the dried product is a spray-dried powder, a freeze-dried powder, a vacuum-dried powder, or a drum-dried powder.

5. The composition of claim 1, wherein the lactic acid bacterium is in a form of a heat-inactivated bacterium.

6. The composition of claim 1, being a dietary composition or a pharmaceutical composition.

7. A method for preventing or treating an allergy in a subject in need thereof, comprising administering an effective amount of Lactobacillus paragasseri to the subject.

8. The method of claim 7, wherein the allergy is a food allergy, an insect allergy, a mold allergy, a pollen allergy, a skin allergy, or a respiratory allergy.

9. The method of claim 7, wherein after the administration, at least one of a serum immunoglobulin level and a level of airway inflammation is decreased in the subject.

10. The method of claim 9, wherein the serum immunoglobulin level is a level of at least one of IgE and IgG1.

11. The method of claim 9, wherein the decreased level of airway inflammation is reduced inflammatory cell infiltration in the subject.

12. The method of claim 9, wherein the decreased level of airway inflammation is reduced mucus production in the subject.

13. The method of claim 7, wherein after the administration, an accumulation level of an inflammatory cell in bronchoalveolar lavage fluid is decreased in the subject.

14. The method of claim 13, wherein the inflammatory cell is a leukocyte or an eosinophil.

15. The method of claim 7, wherein after the administration, a level of at least one cytokine is altered in the subject.

16. The method of claim 15, wherein the cytokine is a proinflammatory cytokine or an anti-inflammatory cytokine.

17. The method of claim 15, wherein the cytokine is at least one of a Th17 cytokine, a Treg cytokine, a Th1 cytokine, and a Th2 cytokine.

18. The method of claim 17, wherein the Th17 cytokine is IL-17A, the Treg cytokine is IL-10, the Th1 cytokine is at least one of IFN-γ and IL-12, and the Th2 cytokine is at least one of IL-4, IL-5, and IL-13.

19. The method of claim 15, wherein the cytokine alteration modulates at least one of a Th1 immune response and a Th2 immune response.

20. The method of claim 19, wherein the cytokine alteration modulates the immune response towards Th1.
